(19) United States
(12) Patent Application Publication
(10) Pub. No.: US 2020/0037982 A1
(43) Pub. Date: Feb. 6, 2020

(54) INTRAVASCULAR FLOW AND PRESSURE MEASUREMENTS

(71) Applicant: KONINKLIJKE PHILIPS N.V., EINDHOVEN (NL)

(72) Inventors: Arjen VAN DER HORST, TILBURG (NL); Ameet Kumar JAIN, BOSTON, MA (US)

(21) Appl. No.: 16/498,682

(22) PCT Filed: Mar. 30, 2018
(86) PCT No.: PCT/EP2018/058272

§ 371 Date: Sep. 27, 2019

Related U.S. Application Data
(60) Provisional application No. 62/479,368, filed on Mar. 31, 2017.

(51) Int. Cl. A61B 8/06; A61B 5/06; A61B 8/12; A61B 8/04; A61B 8/00; A61B 8/08

(52) U.S. Cl. CPC A61B 8/06; A61B 5/062; A61B 8/488; A61B 8/04; A61B 8/463; A61B 8/12

(57) ABSTRACT

The present disclosure describes methods and systems of generating a functional flow map of a bodily structure. Methods may involve providing an intraluminal device configured for insertion into a bodily structure that is within a tracked field. The intraluminal device may include a sensor configured to obtain one or more functional flow measurements and configured to receive a signal or cause a disturbance in the tracked field. The received signal or disturbance of the sensor may be used to track one or more positions of the intraluminal device within the bodily structure. The sensor may also be used to obtain the functional flow measurements at the tracked positions. Based on the tracked positions and functional flow measurements, the functional flow map of the bodily structure may be generated.

### RELATED APPLICATION

This application claims the benefit of and priority to U.S. Provisional Application No. 62/479,368, filed Mar. 31, 2017, which is incorporated by reference in its entirety.

### FIELD OF THE INVENTION

The present invention is directed to systems and methods for obtaining hemodynamic measurements and mapping the measurements to specific locations within a bodily structure. More particularly, the present invention is directed to an ultrasound system and method for obtaining blood pressure and/or flow measurements using an intraluminal device and mapping the measurements to specific intraluminal locations using a tracking system to generate a functional flow map of a bodily structure.

### BACKGROUND

Assessing the hemodynamic significance of cardiovascular and peripheral vascular disease by intravascular pressure and/or flow measurement has proven beneficial to guide treatment of atherosclerotic disease. For example, in coronary arteries, using a pressure sensor mounted on a guide-wire/catheter to obtain intraluminal blood pressure measurements is currently the standard of care for assessing cardiovascular disease. However, the location of the pressure sensor must be co-registered with the intraluminal pressure readings according to such methods, which may be difficult. Obtaining accurate intraluminal blood flow velocity measurements using catheters/guide-wires may be even more difficult because the velocity profile of a given blood vessel is typically dependent on vessel anatomy. Moreover, awareness of the location and/or orientation of the flow sensor with respect to the vessel is critical for obtaining reliable flow measurement data, especially using current ultrasound guide-wires, which generally determine blood flow velocity in the direction of the wire instead of the direction of the vessel. Existing guide-wires thus often fail to provide accurate measurements of blood flow and blood pressure. Accordingly, techniques for more accurately and reliably measuring blood flow and blood pressure at localized positions within blood vessels may be desired.

### SUMMARY

Provided herein are methods, systems, and apparatuses for obtaining hemodynamic measurements within a bodily structure, such as the lumen of a blood vessel, and mapping the measurements to specific intraluminal locations using an external tracking system. Tracking data gathered by the external tracking system may be used to evaluate the quality of the hemodynamic measurements obtained within the lumen, which may be obtained using one or more sensors included on an intraluminal device, e.g., a guide wire. In certain embodiments, the tracking data is acquired from the same sensors used for the hemodynamic measurements. For example, certain measurements may be discarded based on the position and/or orientation of the sensor relative to the intraluminal walls of the bodily structure. One or more processors may be utilized to combine the externally-acquired tracking data with the intraluminal hemodynamic data and create a functional flow map that superimposes both data types. In some examples, the bodily structure containing the sensor may also be imaged and the resulting image(s) overlaid on the functional flow map such that intraluminal measurements are displayed on an image of a bodily structure at the locations the measurements are obtained. Hemodynamic data acquired within the bodily structure may include measurements of intraluminal blood pressure, blood flow velocity, and/or blood flow direction. Hemodynamic data may also be estimated externally using a tracking system, for example via Doppler flow imaging, and utilized in conjunction with corresponding intraluminal measurements to adjust inaccurate and/or misaligned measurements. Tracking systems that may be implemented to perform the methods described herein may include ultrasound and/or electromagnetic tracking systems, each system configured to generate a tracked field within which the position and/or orientation of the intraluminal device may be monitored. In certain embodiments, tracking is based on when a sensor receives a signal from a disturbance using time-of-flight measurements, e.g., positions within a tracked field are based on the time it takes for the external signal or disturbance to be received by the sensor.

In accordance with some examples, a method may involve providing an intraluminal device configured for insertion into a bodily structure that is within a tracked field. The intraluminal device may include one or more sensors, and at least one sensor is configured to obtain one or more functional flow measurements and configured to receive a signal or cause a disturbance in the tracked field. The method may further involve using the received signal or disturbance of the sensor to track one or more positions of the intraluminal device within the bodily structure, using the sensor to obtain the functional flow measurements at the tracked positions, and generating a functional flow map of the bodily structure based on the tracked positions and functional flow measurements.

In some examples, the functional flow measurements may include at least one of blood pressure or blood flow velocity. In various implementations, the method may be performed in real time as the intraluminal device is moved through the tracked field. In some embodiments, the tracked field may be generated by transmitting ultrasound toward the sensor, where the sensor includes an ultrasound receiver, and where using the received signal involves performing one-way beamforming of the received signal.

Some example methods may further involve providing an indication of quality of the functional flow measurements obtained using the sensor. The indication of quality may be based, at least in part, on the tracked position of the sensor in relation to the tracked field. In some embodiments, the tracked position may include the proximity of the sensor to an intraluminal wall, the angle between the sensor and the intraluminal wall, and/or the level of movement of the sensor relative to the intraluminal wall.

In some embodiments, generating the functional flow map may involve rejecting a measured blood pressure or a measured blood flow velocity associated with a quality value below a threshold quality value. In addition or alternatively, generating the functional flow map may involve combining blood pressure or blood flow velocity measurements obtained using the sensor with flow velocity estimates derived via an external ultrasound system. In some implementations, using the sensor to obtain the functional flow measurements may involve transmitting and receiving intraluminal ultrasound signals at the sensor. The method may further involve displaying an image including the functional flow map overlaid onto an image of the bodily structure.

In accordance with some examples, a system may include an intraluminal device configured for insertion into a bodily structure within a tracked field. One or more sensors may be positioned on the intraluminal device, where at least one sensor is configured to obtain one or more functional flow measurements and configured to receive a signal or cause a disturbance in the tracked field. The system may further include a tracking system communicatively coupled to the sensor to generate tracking data responsive to the received signal or disturbance caused by the sensor. The system may further include one or more processors in communication with the sensor and the tracking system. The one or more processors may be configured to: use the received signal or disturbance of the sensor to track one or more positions of the intraluminal device within the bodily structure, use the sensor to obtain the functional flow measurements at the tracked position, and generate a functional flow map of the bodily structure based on the tracked positions and functional flow measurements. The functional flow measurements may include at least a blood pressure and/or blood flow velocity. In some examples, the system may further include a display in communication with the one or more processors, where the one or more processors are configured to cause the display to display an image including the functional flow map overlaid onto an image of the bodily structure. In some embodiments, one or more processors are configured to cause the display to display an indication of a quality of the blood pressure or blood flow velocity measurements obtained using the sensor, where the indication of quality of the blood pressure or blood flow velocity measurements is based, at least in part, on the tracked position. The one or more processors may be further configured to ignore blood pressure or blood flow velocity measurements associated with a quality value below a threshold quality value when generating the functional flow map.

In some embodiments, the tracking system may include an ultrasonic tracking system including an ultrasound transmitter configured to transmit ultrasound toward the sensor, where the sensor includes an ultrasound receiver, and where the ultrasonic tracking system is configured to localize the position of the ultrasound receiver by performing one-way beamforming of signals received by the ultrasound receiver when placed within a field of view of the ultrasound transmitter. In some examples, the tracking system may be provided by an ultrasonic array of an ultrasound imaging system configured to generate an ultrasound image including a B-mode image of the bodily structure overlaid with the functional flow map. The ultrasound imaging system may be configured to generate an ultrasound image further including the tracked position of the sensor, updated in real-time, in the ultrasound image. In some examples, the tracking system may include an ultrasound system configured to generate color-flow Doppler or vector flow images, where the one or more processors are configured to combine the one or more functional flow measurements obtained using the sensor with flow velocity estimates derived by the ultrasound system to generate the functional flow map.

In additional or alternative implementations, the tracking system may include an electromagnetic (EM) tracking system including a field generator configured to generate an EM field, where the sensor includes one or more sensor coils, and where the EM tracking system is configured to localize the tracked position of the one or more sensor coils responsive to a disturbance of the EM field caused by the one or more sensor coils when placed within the EM field.

### BRIEF DESCRIPTION OF THE DRAWINGS

FIG. 1 is a block diagram of a flow map generation system in accordance with the principles of the present disclosure.

FIG. 2 is an illustration of an intraluminal device equipped with a sensor positioned near the center of a blood vessel in accordance with the principles of the present disclosure.

FIG. 3 is an illustration of an intraluminal device equipped with a sensor positioned near a turning point of a blood vessel in accordance with the principles of the present disclosure.

FIG. 4 is an illustration of a moving intraluminal device equipped with a sensor positioned near the center of a blood vessel in accordance with the principles of the present disclosure.

FIG. 5 is a block diagram of another flow map generation system in accordance with the principles of the present disclosure.

FIG. 6 is a block diagram of an ultrasound tracking system in accordance with the principles of the present disclosure.

FIG. 7 is block diagram of a flow map generation method in accordance with principles of the present disclosure.

### DETAILED DESCRIPTION

The following description of certain exemplary embodiments is merely exemplary in nature and is in no way intended to limit the invention or its applications or uses. In the following detailed description of embodiments of the present systems and methods, reference is made to the accompanying drawings which form a part hereof, and in which are shown by way of illustration specific embodiments in which the described systems and methods may be practiced. These embodiments are described in sufficient detail to enable those skilled in the art to practice the presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

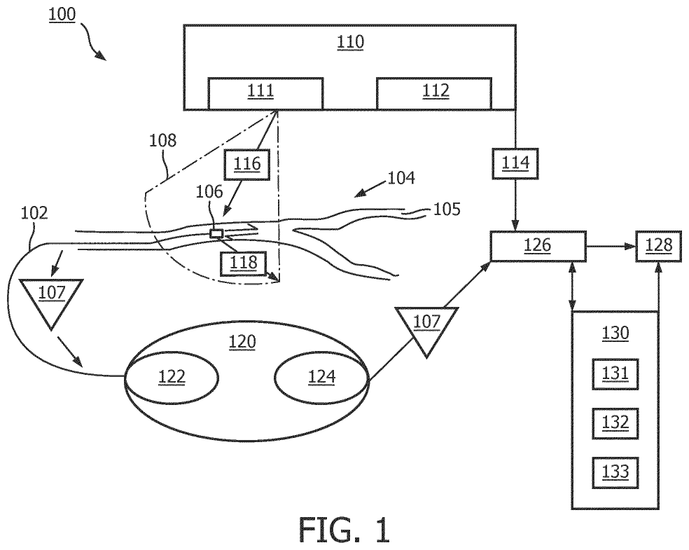

FIG. 1 shows an example system 100 configured to generate a functional flow map of a bodily structure in accordance with the present disclosure. As shown, the system 100 may include an intraluminal device 102, e.g., a catheter, micro-catheter, or guide-wire, configured for insertion into a bodily structure 104, such as a blood vessel, which defines an internal lumen 105. The intraluminal device 102 may include one or more sensors 106 configured to obtain various functional flow measurements, e.g., blood flow velocity and/or blood pressure, in the form of hemodynamic, intraluminal data 107. The functional flow measurements may be obtained within a tracked field 108 generated by an external tracking system 110. At least one of the sensors 106 is also configured to receive a signal or disturbance generated by the external tracking system 110. The tracking system 110 shown in FIG. 1 includes a tracking field generator 111 and a tracking processor 112, both of which may be communicatively coupled to the sensor 106. The tracking system 110 may be configured to generate tracking data 114 responsive to detected movement of the sensor 106, for example responsive to disturbances 118 caused by the sensor 106 within the tracked field 108. In other embodiments, the tracking system 110 may transmit signals 116, such as ultrasound signals, toward the sensor 106, which may include an ultrasound receiver. The sensor 106 may generate signals responsive to the detected ultrasound and the signals may be transmitted (e.g., via a wired or wireless connection) to the tracking system 110 or to the processor 126 (such as via the wired connection used to transmit intraluminal data 107). The tracking system 110 may generate tracking data 114 which may be used to track the sensor 106 in relation to the bodily structure. For example, the tracking data 114 generated by the tracking system 110 may embody information regarding the position and/or orientation of the sensor 106, which may be collected in real time as the intraluminal device 102 is moved through the bodily structure 104. In embodiments in which the sensor 106 communicates tracking signals directly to the processor 126, the processor 126 may be configured to determine the position and/or orientation of the sensor 106 relative to the bodily structure 104. For example, the processor may be configured to generate the tracking data 114 based on the signals from sensor 106 and/or additional information (e.g., information about the ultrasound tracking pulses) transmitted from tracking system 110. Other arrangements of tracking sensors in relation to a tracking system and/or processor 126 may be used.

As further shown, the intraluminal device 102 may be coupled with a device system 120 which may include a device controller 122 and a device processor 124 configured for operating the intraluminal device 102 and processing the intraluminal data 107 it collects, respectively. Communicatively coupled with both the tracking processor 112 and the device processor 124 is an integrated processor 126. The integrated processor 126 may be configured to receive and process tracking data 114 received from the tracking system 110 and intraluminal data 107 received from the device system 120. By compiling data from both informational sources, the integrated processor 126 may be configured to combine positional information of the sensor 106 with functional flow measurements obtained within the lumen 105, thereby generating a functional flow map 128 that includes functional flow measurements mapped to specific positions within the bodily structure 104. The system shown in FIG. 1 also includes a display 130, which may be communicatively coupled with the integrated processor 126, and may include an interactive user interface 131. The display 130 may be configured to display the functional flow map 128 overlaid onto an image 132, e.g., B-mode, of the bodily structure 104. In some examples, the display 130 may be further configured to display an indication of quality 133 of one more functional flow measurements.

The integrated processor 126 may be configured to process data received from both the sensor 106 and the tracking system 110 in multiple ways. The integrated processor 126 may be formed from one or a plurality of processers. For example, as stated above, the integrated processor 126 may be configured to generate a functional flow map 128. The functional flow map 128 may include various flow and/or pressure measurements collected from within the lumen 105 of the bodily structure 104, each measurement corresponding to a particular location at which the measurement was obtained. In one example, the functional flow map 128 may include blood flow velocity measurements mapped to one or more locations within a blood vessel. As the intraluminal device 102 is moved through the bodily structure 104, new velocity measurements collected by the sensor 106 may be added to the map 128 at the discrete locations of their detection. In addition or alternatively, the functional flow map 128 may contain blood pressure readings obtained by the sensor 106 at various locations within a blood vessel. In yet another example, blood flow directional data may be obtained by the sensor 106 and mapped to different locations within the functional flow map 128. Depending on the functionality of the sensor 106, or the number of sensors coupled with a particular intraluminal device 102, two or more different types functional flow measurements may be included on the functional flow map 128. Such measurements may be displayed, via the display 130, simultaneously or individually. Under the control of a user, the display 130 may be configured to switch between multiple versions of the functional flow map 128. For instance, a user desiring to view only data regarding blood flow velocity may select an option, for example at the user interface 131, for viewing a functional flow map 128 comprising such data. Another selectable option may comprise blood pressure data only, while another may comprise two or more data types.

Obtaining functional flow measurements via the sensor 106 and mapping these measurements to specific intraluminal locations via the tracking system 110 may facilitate improved data gathering and interpretation. For example, blood pressure, fractional flow reserve (“FFR”), instantaneous wave free ratio (“iFR”), blood flow velocity, blood flow direction, volumetric blood flow, coronary flow reserve (“CFR”), flow resistance, and/or micro-circulation, among other hemodynamic parameters, may fluctuate at different locations within a blood vessel. The ability to measure these parameters using currently marketed guide-wires/catheters also varies at different locations. For example, a sensor oriented parallel to the surrounding intraluminal walls may collect more accurate data regarding blood flow velocity than the same sensor oriented perpendicular to an intraluminal wall. Accordingly, by considering functional flow measurements together with the location at which the measurements are obtained, the quality or accuracy of the measurements can be assessed. As discussed herein, the accuracy of intraluminal data 107 may be corrected by merging such data with externally-obtained tracking data 114. In addition or alternatively, intraluminal data 107 may be screened based on the tracking data 114 obtained via the tracking system 110.

The integrated processor 126 depicted in FIG. 1 comprises a singular component which is coupled with two separate processors, however, the configuration of the integrated processor 126 may vary. For example, the integrated processor 126 may include two or more processors. In some examples, integration of intraluminal data 107 with tracking data 114 may not be performed by a distinct processing component. Instead, data integration may occur within the same processor used to process tracking data 114 and/or intraluminal data 107. Accordingly, the device processor 124 and/or the tracking processor 112 may be configured to generate the functional flow map 128 in some examples. In various embodiments, a distinct device processor 124 and tracking processor 112 may be omitted altogether, such that the integration processor 126 performs the initial processing, as well as the ultimate merging, of data received from the tracking system 110 and the device system 120.

The manner by which the integrated processor 126 is configured to process various functional flow measurements, e.g., blood pressure and/or blood flow velocity, in tandem with the tracking data 114 may vary. In some embodiments, the integrated processor 126 may be configured to determine a quality indication 133 of one or more functional flow measurements obtained using the sensor 106. The quality indication 133 may be based, at least in part, on the tracked position of the sensor 106 and may be displayed on the display 130 in real time as the measurements are being collected. The quality indication 133 may improve when the sensor 106 is positioned within relatively straight portions of a blood vessel relative to curved portions, especially near particularly tight corners, for example, where the sensor 106 may be more likely to be facing an intraluminal wall. The quality indication 133 may be displayed in a binary fashion, such that a given functional flow measurement is either deemed “quality” or “not quality,” or the quality indication 133 may be continuously adjusted on a relative scale from “high quality” to “low quality.” According to such embodiments, quality measurements may be evaluated on a frame-by-frame basis as tracking data 114 and intraluminal data 107 is obtained, for example via ultrasound.

In some examples, the integrated processor 126 may be configured to reject, ignore, or otherwise filter functional flow measurements associated with a quality value below a threshold quality value when generating the functional flow map 128. In this manner, the functional flow map 128 may selectively include only functional flow measurements deemed to be of sufficient quality. Measurements satisfying the quality threshold applied by the integrated processor 126 may be interpolated where data points corresponding to intervening intraluminal locations and/or time points are excluded. Various parameters may be evaluated by the integrated processor 126 to determine whether the functional flow measurements satisfy a threshold quality value. For example, the tracked position of the sensor 106 may include information regarding the proximity of the sensor 106 to an intraluminal wall, the angle between the sensor 106 and an intraluminal wall, and/or a level of movement of the sensor 106 relative to the intraluminal wall. One or more of these parameters may impact the quality of the measurements obtained by the sensor 106. For instance, the quality may decrease at positions closer to an intraluminal wall. In some embodiments, the integrated processor 126 may be configured to automatically determine, based on the tracking data 114 received from the tracking system 110, whether a distal tip portion of the intraluminal device 102, which may be coupled with a sensor 106, is positioned in the radial center of a blood vessel, near the blood vessel wall, or somewhere in between. Functional flow measurements obtained when the sensor 106 is positioned near the wall may be rejected, while positions closer to or at the center may be accepted for further processing and/or inclusion within the functional flow map 128. Similarly, quality may decrease if the sensor 106 is moving toward or away from an intraluminal wall, for example if the sensor is oscillating due to turbulent blood flow and/or movement of the intraluminal device 102 by a user.

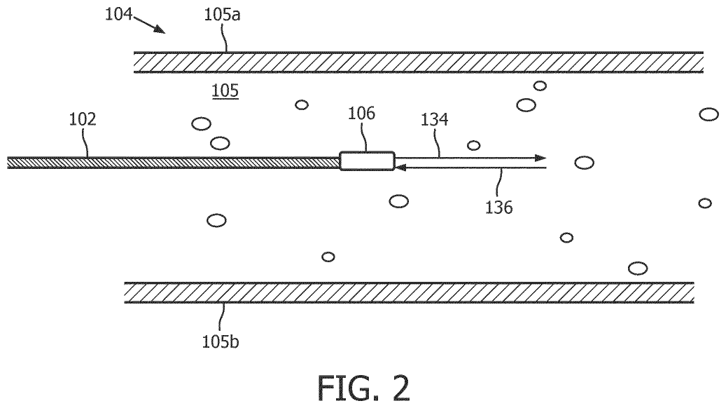

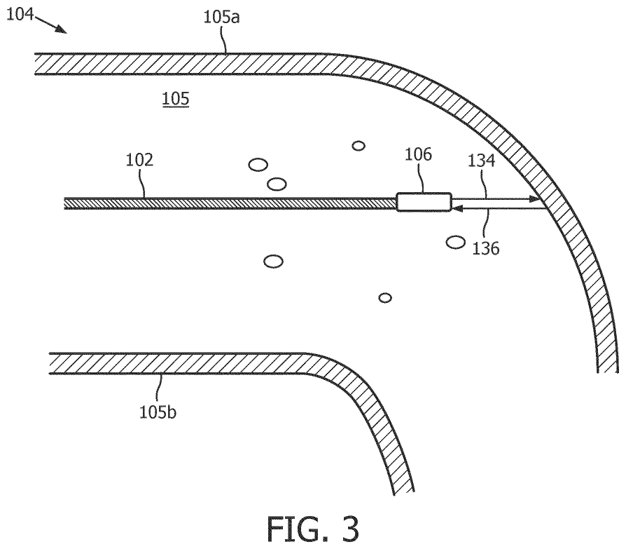

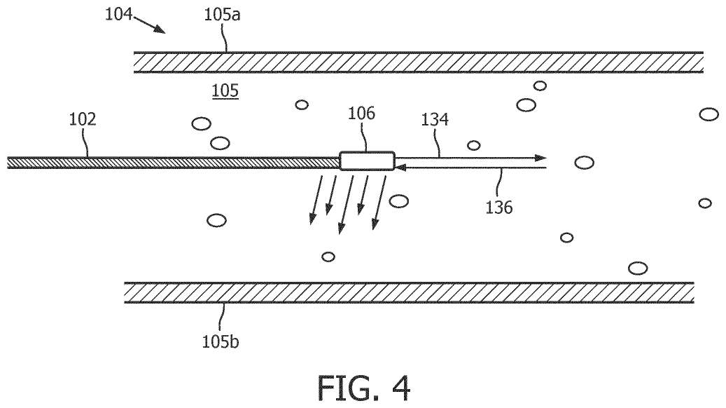

Example scenarios in which functional flow measurements may be rejected or accepted by the integrated processor 126 are illustrated in FIGS. 2-4. Each figure depicts an intraluminal device 102 coupled with a sensor 106 positioned within the lumen 105 of a bodily structure 104, represented as a blood vessel. For explanatory purposes, the blood vessel 104 is shown as including two parallel intraluminal walls 105a and 105b defining the intraluminal space 105; however, the blood vessel 104 comprises only one, cylindrical, intraluminal wall. In FIG. 2, the sensor 106 is positioned near the center of the blood vessel 104, approximately equidistant from both intraluminal walls 105a, 105b. Functional flow measurements gathered by the sensor 106 from this position and orientation may be accepted by the integrated processor 126 and displayed on a functional flow map 128. By contrast, FIG. 3 illustrates a scenario in which functional flow measurements gathered by the sensor 106 may be rejected by the integrated processor 126. In particular, the sensor 106 is facing toward intraluminal wall 105a and away from intraluminal wall 105b. In this orientation, the sensor 106 may collect inaccurate measurements. As such, these measurements may be excluded from the functional flow map 128. FIG. 4 depicts yet another scenario in which the integrated processor 126 may be configured to reject the intraluminal data 107 gathered by the sensor 106. In FIG. 4, the sensor 106 is moving vertically in the direction of the arrows, toward intraluminal wall 105b. The sensor 106 may be oscillating up and down, alternately approaching intraluminal wall 105a and 105b in quick succession. Such movement of the sensor 106 may prevent it from obtaining accurate measurements, especially regarding flow velocity. As such, intraluminal data 107 obtained in this orientation may also be excluded.

Intraluminal data 107 regarding functional flow measurements obtained via the sensor 106 may be rejected, or deemed to be of lesser quality, for numerous additional reasons. For instance, certain locations within a bodily structure 104 may be preemptively avoided, and thus intraluminal data 107 obtained in these locations may be automatically rejected by the integrated processor 126. Intraluminal data 107 may also be rejected if secondary flows are detected within the lumen 105, which may occur particularly frequently near vessel bifurcations and/or stenosis. Secondary flows may be detected manually or automatically by the tracking system 110 and communicated to the integrated processor 126 to aid in data filtration.

The intraluminal device 102 and the sensor 106 may comprise various configurations and/or functionalities. Sensors 106 may be permanently attached, integrally formed with, or reversibly coupled with an intraluminal device 102. The window within which a sensor 106 obtains intraluminal data 107 may be localized and variable in size. For example, the measurement window size of the sensor 106 may range from about 1 mm to about 15 mm, about 2 mm to about 12 mm, about 3 mm to about 9 mm, about 4 to about 8 mm, or about 6 mm. As shown in FIGS. 1-4, only one sensor 106 may be included on an individual intraluminal device 102 in some embodiments. According to some examples, a single sensor 106 may be configured to alternate between different operational modes. For instance, the sensor 106 may comprise an ultrasound receiver communicatively coupled with an external ultrasound tracking system. In such examples, the sensor 106 may be configured to alternate between a receive mode and a transmittal mode. In the receive mode, the sensor 106 may operate to receive externally-generated ultrasound signals, and in the transmittal mode, the sensor 106 may be configured to transmit ultrasound signals in the lumen 105. In additional or alternatively, the sensor 106 may be time sliced to alternate between a tracking mode and a measurement mode. In the tracking mode, for example, the sensor 106 may transmit and/or receive ultrasound signals, while in the measurement mode, the sensor 106 may monitor intraluminal blood pressure. Alternatively, two or more sensors 106 may be included on a single intraluminal device 102. Where two or more sensors are included on a single device, the sensors may be offset from each other by various distances along the length of the device. In some examples, each sensor may be configured to perform a distinct function, which may depend in part on the variety of tracking system utilized. For instance, a first sensor may be a tracking sensor configured to receive ultrasound signals transmitted toward the sensor from an external ultrasound transmitter, while a second sensor may be configured to determine one or more functional flow measurements within a lumen of a bodily structure, such as blood pressure.

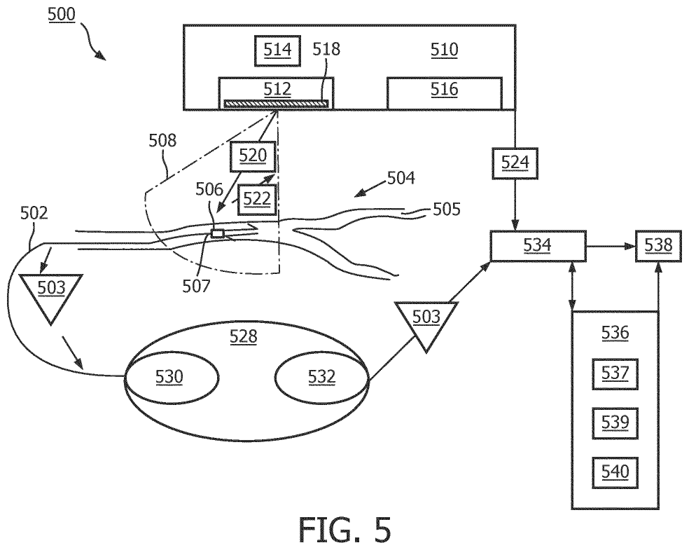

In some embodiments, the sensor 106 may be configured to obtain intraluminal data 107 regarding blood flow. Such a sensor 106 may comprise an ultrasonic transducer, e.g., a lead zirconate titanate (“PZT”) transducer, a capacitive micromachined ultrasonic transducer (“CMUT”), or a single crystal transducer, and may be configured to measure blood flow velocity, for example via Doppler flow, by transmitting ultrasound signals or beams 134 into the lumen 105 of the bodily structure 104 and receiving signals 136 responsive to the transmitted signals 134 (as shown in FIGS. 2-4). As described in greater detail below with reference to FIG. 5, the same sensor 106 may also be configured to receive ultrasound signals transmitted into the bodily structure 104 from an external ultrasound device.

In some embodiments, the sensor 106 may be configured to obtain intraluminal data 107 regarding blood pressure. Such a sensor 106 may be configured to measure blood pressure via various techniques, including fractional flow reserve (“FFR”) and/or instant wave-free ratio FFR (“iFR”), within the lumen 105. Sensors configured to obtain pressure data may be piezo-sensitive and capacitive, and may be used to locate the position of the sensor in an externally-generated, tracked field 108. Functional flow maps 128 comprising blood pressure measurements may include different colors to represent different pressures measured at different locations. Sensors equipped to measure blood pressure may be utilized pursuant to various pullback measurement techniques, which may generally involve detecting pressure gradients within a bodily structure 104 as an intraluminal device 102, and thus the blood pressure sensor 106 coupled thereto, is moved through the bodily structure. Some embodiments may further involve electrocardiogram-gating of blood pressure measurements such that pressure readings are displayed as a function of the heart cycle. According to such embodiments, pressure measurements may be obtained during systole and diastole, with intervening values interpolated via one or more of the processors shown in FIG. 1.

In some examples, the sensor 106 may be configured to facilitate tracking. For example, the sensor 106 may include one or more sensors, in some examples an array of sensors, to receive signals or disturbances transferred through the body. The signals transferred through the body may be ultrasound, mechanical, electromechanical, etc. Specific embodiments may involve tracking the sensor 106 for example as described in U.S. Pat. Pub. No.: 2016/0317119 (Maraghoosh), which is incorporated by reference in its entirety herein. According to such embodiments, the sensor 106 may include one or more sensors (e.g., an ultrasound sensor) responsive to signals generated from an external tracking system 110. The external tracking system may be operatively associated with the intraluminal sensor 106 to track a position of the sensor 106. The tracking system 110 may include a processor, such as tracking processor 112, which may be configured to determine a position and/or orientation of the sensor 106 according to the signals generated by the sensor 106 and received by the external tracking system 110. In some embodiments the sensor 106 may be an ultrasonic receiver configured to detect ultrasound waves. The sensor 106 may generate signals responsive to the detected waves, and the signals may be communicated to the processor 112 of the tracking system for determining the relative position and/or orientation of the sensor 106 in relation to the source of the ultrasonic waves. In such embodiments, one-way beamforming (e.g., reflecting the one-way time of flight between the source and sensor) may be used for determining the position of sensor 106. In other embodiments, the sensor 106 may be an ultrasonic transmitter configured to generate ultrasound toward an external receiver (e.g., an imaging array). The relative position and/or orientation of the sensor 106 in relation to the external receiver may thus be determined. According to examples of the present disclosure, the type of tissue surrounding the sensor 106 may also be classified responsive to the signals received at the sensor 106. In additional embodiments, different arrangements of ultrasound tracking sensors (e.g., sensor 106) may be used, which may employ one-way or two-way beamforming to determine the position and/or orientation of the sensor at any given time. Furthermore, non-ultrasonic sensors (e.g., EM tracking sensors) may be used in other examples.

The intraluminal device 102 may include one or more sensors 106 configured to measure one or more hemodynamic characteristics, including blood flow velocity, blood pressure, and/or blood flow direction. Example intraluminal devices that may be implemented in the system 100 include FLOWIRE, VERRATA, and/or COMBOWIRE, each by Koninklijke Philips Volcano (“Philips”). The intraluminal device 102 may be configured for manual steering within the bodily structure 104 in some examples. Movement of the device 102 could also be performed robotically, with image guidance provided by an ultrasound tracking system, for example.

The type of tracking system 110 included in the system 100 may vary in different embodiments. For example, the tracking system 110 may comprise an electromagnetic tracking system. According to such examples, the tracking field generator 111 may comprise an electromagnetic field generator configured to generate an electromagnetic field 108 encompassing the bodily structure 104 that contains the intraluminal device 102. The sensor 106 employed may include one or more sensor coils. In operation, the electromagnetic tracking system 110 may be configured to localize the tracked position of the one or more sensor coils responsive to a disturbance within the electromagnetic field caused by the one or more sensor coils when placed within the electromagnetic field. In some examples, the electromagnetic tracking system 110 may be utilized in conjunction with an imaging system, e.g., ultrasound imaging system. Such examples may include at least two sensors 106 mounted at known positions on a single intraluminal device 102. The first sensor may contain coils configured to cause a disturbance within the electromagnetic field 108 generated by the tracking field generator 111, and the second sensor may include an array configured to receive ultrasound signals from an external ultrasound imaging system. The position of the second sensor may be registered to the position of the first sensor by the tracking system 110 and the imaging system to determine the position and/or orientation of the sensors on the intraluminal device, for example as described in U.S. Pat. Pub. No.: 2015/0269728 (Parthasarathy), which is incorporated by reference in its entirety herein. In further examples, the EM tracking system 110 may not include the ultrasound sensor and may be configured to determine the location of the EM sensor, which may be registered to the EM tracking field, based on the movement of the EM sensor within the tracking field.

The functionality of a given tracking system 110 may impact its degree of input within the overall system 100. For example, the tracking system 110 may also comprise an ultrasound tracking system, which may estimate blood flow characteristics from an external vantage point. FIG. 5 shows an example of such a system in accordance with an embodiment of the present disclosure. Like system 100 shown in FIG. 1, the system 500 includes an intraluminal device 502 positioned within a bodily structure 504, such as a blood vessel having a lumen 505. The intraluminal device 502 includes at least one sensor 506 that includes an ultrasound receiver 507. The sensor 506 may be configured to obtain various functional flow measurements, e.g., blood pressure and/or blood flow velocity, within a tracked field 508 generated by an external ultrasound tracking system 510. In this embodiment, the external ultrasound tracking system 510 includes an external ultrasound probe 512, a probe controller 514, and an ultrasound processor 516, each of which may be coupled within the tracking system. The probe 512 may include an ultrasound sensor array 518 configured to transmit ultrasound signals 520 into the bodily structure 104 and receive signals 522 responsive to the transmitted signals. The ultrasound tracking processor 516 may be configured to generate tracking data 524 responsive to the received signals 522. Tracking data 524 generated by the ultrasound tracking system 510 may embody information regarding the position and/or orientation of the sensor 506, along with externally-acquired functional flow data. As in system 100, the intraluminal device 502 may be coupled with a device system 528 which may include a device controller 530 and a device processor 532 configured for operating the intraluminal device 502 and processing the intraluminal data 503 it collects, respectively. Communicatively coupled with both the ultrasound tracking processor 516 and the device processor 532 is an integrated processor 534, configured to receive and process data from the ultrasound tracking system 510 and the device system 528. The system 500 may be coupled with a display 536, which may be configured to display an image 537 of the bodily structure, which may be merged with a functional flow map 538 generated by the integrated processor 534. A user interface 539 and an indication of quality 540 may also be included and/or displayed by the display 536.

With the ultrasound tracking system 510, tracking the position and/or orientation of the sensor 506 may further involve imaging the sensor 506 and in some examples, one or more aspects of the surrounding features of the bodily structure 504. In some implementations, the ultrasound tracking system 510 may be configured to localize the position of the ultrasound receiver 507 by performing one-way beamforming of signals received at the receiver 507, i.e., the transmitted signals 520, when placed within the field of view of the ultrasound probe 512, for example as described in US. Pat. Pub. No.: 2013/0041252 (Vignon), which is incorporated by reference in its entirety herein. Such implementations may involve emitting one or more ultrasound pulses from the external ultrasound tracking system 510. Each pulse may be received by the ultrasound receiver 507 on the sensor 506. Based on the time-of-flight from the emission of the pulse until its receipt at the receiver 507, the distance of the receiver 507, and thus the sensor 506, from the external ultrasound tracking system 510 may be determined. In embodiments, the ultrasound tracking system 510 may be provided by an ultrasonic array of an ultrasonic imaging system configured to generate ultrasound images 537 of the bodily structure 504. The images 537 may be of a variety of different types, e.g., B-mode, Doppler, vector flow, and/or raw-signal display, to name a few. One or more of these images 537 may be overlaid on the functional flow map 538 generated by the integrated processor 534. The ultrasound images 537 may include the tracked position of the sensor 506 and may be updated in real time. In some examples, multiple image types may be integrated into the same functional flow map 538, such that the map contains, for instance, a B-mode and Doppler images overlaid on an FFR pull-back map. Possible ultrasonic imaging systems may include, for example, a mobile system such as LUMIFY by Philips, or SPARQ and/or EPIQ, also produced by Philips.

Externally-acquired tracking data 524 that includes functional flow data gathered via the ultrasound tracking system 510 may be used to improve the accuracy of intraluminal data 503 gathered at the sensor 506. In particular, the externally-acquired functional flow data may be used to enrich the intraluminal data by correcting misaligned data or otherwise combining the intraluminal data with the externally-acquired data. For instance, in addition to collecting tracking data 524 regarding the position/orientation of the sensor 506, the ultrasound tracking system 510 may be configured to estimate the flow velocity and/or flow direction of the blood within the bodily structure 504. In specific embodiments, the ultrasound tracking system 510 may be configured to generate color-flow Doppler or vector flow images. According to such embodiments, the integrated processor 534 may be configured to combine the one or more functional flow measurements obtained using the sensor 506 with flow estimates derived by the ultrasound tracking system 510. This compiled data may be merged into the functional flow map 538. In various embodiments, the ultrasound tracking system 510 may be configured to acquire information regarding blood vessel locations, blood vessel size, Doppler-based flow information, 3D information, and/or vector flow information. Any or all of these information types may be used to supplement and/or modify intraluminal data 503 acquired at the sensor 506 to provide improved flow information for inclusion in the functional flow map.

In particular embodiments, tracking data 524 gathered from the ultrasound tracking system 510 may be integrated with intraluminal data 503 as part of an improved flow calculation model. A flow calculation model my feed local intraluminal data into external Doppler and/or vector flow estimates, or vice versa. Different external imaging modes of the ultrasound tracking system may also be utilized to improve the accuracy of the external flow estimates. The cross-sectional area of the bodily structure at the location of the sensor may also be estimated and incorporated into the overall flow estimate, which may be generally calculated by multiplying the cross-sectional area of a specific location within the bodily structure by the average velocity measured at that location. In some examples, 3D volumetric flow calculations obtained via a 3D ultrasound probe may be used to further improve volumetric flow calculations.

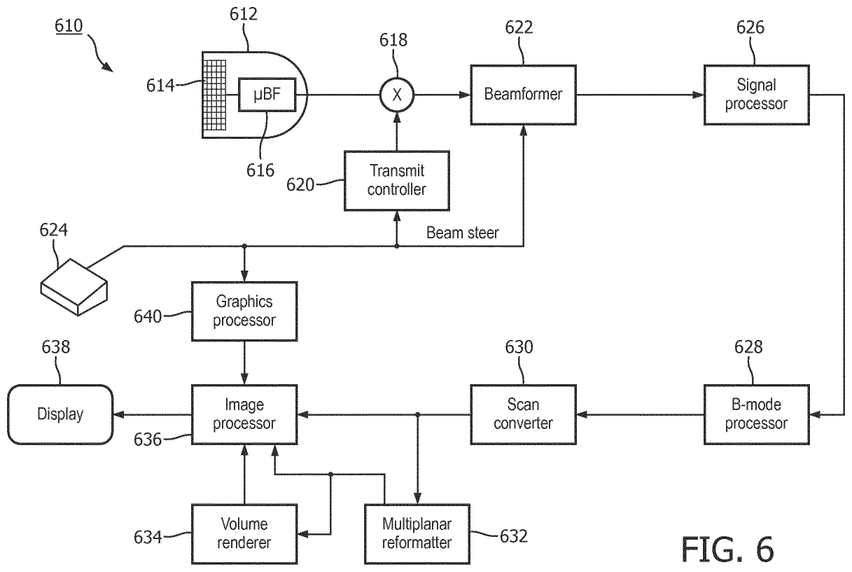

FIG. 6 is a block diagram of an external ultrasound tracking system 610 configured to obtain ultrasound images in accordance with the principles of the present disclosure. The ultrasound tracking system 610 may be incorporated into a system for generating a functional flow map, such as system 510. The images acquired via the ultrasound tracking system 610 may be merged with a functional flow map, such that a variety of information, e.g., blood pressure and/or flow velocity, obtained at specific locations within a blood vessel may be displayed on an image of the blood vessel at the locations where the measurements were collected. The ultrasound tracking system 610 includes additional components, not shown in FIG. 5, which may be included within a tracking system configured to detect a sensor coupled with an intraluminal device. For example, processing operations performed by one or more of the processors mentioned above, such as ultrasound processor 516, device processor 532 and/or integrated processor 534, may be implemented in and/or controlled by one or more of the processing components shown in FIG. 6, including for example, the B mode processor 628, volume renderer 634, and/or image processor 636. In addition, one or more of the components shown in FIG. 6 may be physically, operatively, and/or communicatively coupled with additional components of a system for generating a functional flow map, including for example, the integrated processor 534 and/or the display 536.

In the ultrasound tracking system 610 of FIG. 6, an ultrasound probe 612 includes a transducer array 614 for transmitting ultrasonic waves at a sensor on an intraluminal device and receiving echo information. A variety of transducer arrays are well known in the art, e.g., linear arrays, convex arrays or phased arrays. The transducer array 614, for example, can include a two dimensional array (as shown) of transducer elements capable of scanning in both elevation and azimuth dimensions for 2D and/or 3D imaging. The transducer array 614 is coupled to a microbeamformer 616 in the probe 612 which controls transmission and reception of signals by the transducer elements in the array. In this example, the microbeamformer is coupled by the probe cable to a transmit/receive (T/R) switch 618, which switches between transmission and reception and protects the main beamformer 622 from high energy transmit signals. In some embodiments, the T/R switch 618 and other elements in the system can be included in the transducer probe rather than in a separate ultrasound system base. The transmission of ultrasonic beams from the transducer array 614 under control of the microbeamformer 616 is directed by the transmit controller 620 coupled to the T/R switch 618 and the beamformer 622, which receives input from the user's operation of the user interface or control panel 624. One of the functions controlled by the transmit controller 620 is the direction in which beams are steered. Beams may be steered straight ahead from (orthogonal to) the transducer array, or at different angles for a wider field of view. The direction may be altered responsive to movement of an intraluminal device within a bodily structure, for example during implementation of a pull-back method used to acquire blood pressure readings within a blood vessel. The partially beamformed signals produced by the microbeamformer 616 are coupled to a main beamformer 622 where partially beamformed signals from individual patches of transducer elements are combined into a fully beamformed signal.

The beamformed signals are coupled to a signal processor 626. The signal processor 626 can process the received echo signals in various ways, such as bandpass filtering, decimation, I and Q component separation, and harmonic signal separation. The signal processor 626 may also perform additional signal enhancement such as speckle reduction, signal compounding, and noise elimination. The processed signals are coupled to a B mode processor 628, which can employ amplitude detection for the imaging of structures in the body. The signals produced by the B mode processor are coupled to a scan converter 630 and a multiplanar reformatter 632. The scan converter 630 arranges the echo signals in the spatial relationship from which they were received in a desired image format. For instance, the scan converter 630 may arrange the echo signal into a two dimensional (2D) sector-shaped format, or a pyramidal three dimensional (3D) image. The multiplanar reformatter 632 can convert echoes which are received from points in a common plane in a volumetric region of the body into an ultrasonic image of that plane, as described in U.S. Pat. No. 6,443,896 (Detmer). A volume renderer 634 converts the echo signals of a 3D data set into a projected 3D image as viewed from a given reference point, e.g., as described in U.S. Pat. No. 6,530,885 (Entrekin et al.) The 2D or 3D images are coupled from the scan converter 630, multiplanar reformatter 632, and volume renderer 634 to an image processor 636 for further enhancement, buffering and temporary storage for display on an image display 638. The graphics processor 636 can generate graphic overlays for display with the ultrasound images. These graphic overlays can contain, e.g., standard identifying information such as patient name, date and time of the image, imaging parameters, and the like. For these purposes the graphics processor receives input from the user interface 624, such as a typed patient name. The user interface can also be coupled to the multiplanar reformatter 632 for selection and control of a display of multiple multiplanar reformatted (MPR) images.

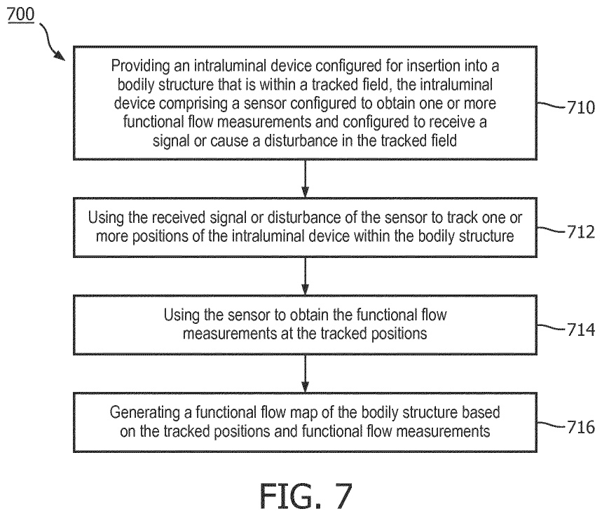

FIG. 7 is block diagram of a flow map generation method 700 in accordance with principles of the present disclosure. The example method 700 of FIG. 7 shows the steps that may be utilized, in any sequence, by the systems and/or apparatuses described herein for generating a functional flow map and/or improving the accuracy of functional flow data obtained within a bodily structure, such as a blood vessel.

At block 710, the method involves “providing an intraluminal device configured for insertion into a bodily structure that is within a tracked field, the intraluminal device comprising a sensor configured to obtain one or more functional flow measurements and configured to receive a signal or cause a disturbance in the tracked field.” In some examples, the functional flow measurements may include blood pressure and/or blood flow velocity.

At block 712, the method involves “using the received signal or disturbance of the sensor to track one or more positions of the intraluminal device within the bodily structure.” Depending on the tracking system utilized, the tracked field may include an electromagnetic field or a field generated by transmitted ultrasound signals. Consequently, the sensor may be tracked in different ways. When an electromagnetic field is employed, the sensor may cause field disturbances detected by the electromagnetic tracking system. Ultrasound tracking systems, by contrast, may track the position of the sensor by transmitted ultrasound signals at the sensor and receiving echoes response to the transmitted signals.

At block 714, the method involves “using the sensor to obtain the functional flow measurements at the tracked positions.” The processes employed by the sensor to obtain functional flow measurements from within the bodily structure may vary. For example, the sensor may include a pressure sensor and/or a flow velocity sensor. A flow velocity sensor may be configured to transmit and receive ultrasound signals from within the lumen of the bodily structure, thereby obtaining velocity information via Doppler flow. The number of sensors, and their position on the intraluminal device, may vary. In some examples, separate sensors may be configured to perform different functions. Where multiple sensors are implemented, one or more may include an ultrasound receiver configured to receive ultrasound signals transmitted from an external ultrasound system.

At block 716, the method involves “generating a functional flow map of the bodily structure based on the tracked positions and functional flow measurements.” The functional flow map may include a variety of measurements, each measurement corresponding to a discrete location within the bodily structure. Embodiments may involve merging the location-specific measurements with Doppler flow and/or B mode images generated by an external ultrasound tracking system, such that the measurements may be viewed on images of the bodily structure at the location each measurement was obtained within the structure. In some examples, the functional flow map may be obtained in real time, for example as the intraluminal device is moved through the bodily structure. Real-time implementation may be reflected by automatic updates to the functional flow map, such that an image of a bodily structure is populated with functional flow measurements as the measurements are obtained.

Of course, it is to be appreciated that any one of the examples, embodiments or processes described herein may be combined with one or more other examples, embodiments and/or processes or be separated and/or performed amongst separate devices or device portions in accordance with the present systems, devices and methods. The above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A method comprising:
providing an intraluminal device configured for insertion into a bodily structure that is within a tracked field, the intraluminal device comprising a sensor configured to obtain one or more functional flow measurements and configured to receive a signal or cause a disturbance in the tracked field;
using the received signal or disturbance of the sensor to track one or more positions of the intraluminal device within the bodily structure;
using the sensor to obtain the functional flow measurements at the tracked positions; and
generating a functional flow map of the bodily structure based on the tracked positions and functional flow measurements.

2. The method of claim 1, wherein the functional flow measurements comprise at least one of blood pressure or blood flow velocity.

3. The method of claim 1, wherein the method is performed in real time as the intraluminal device is moved through the tracked field.

4. The method of claim 1, wherein the tracked field is generated by transmitting ultrasound toward the sensor, wherein the sensor includes an ultrasound receiver, and wherein using the received signal comprises performing one-way beamforming of the received signal.

5. he method of claim 1, further comprising providing an indication of quality of the functional flow measurements obtained using the sensor, wherein the indication of quality is based, at least in part, on the tracked position of the sensor in relation to the tracked field.

6. The method of claim 5, wherein the tracked position comprises at least one of a proximity of the sensor to an intraluminal wall, an angle between the sensor and the intraluminal wall, or a level of movement of the sensor relative to the intraluminal wall.

7. The method of claim 1, wherein generating the functional flow map comprises rejecting a measured blood pressure or a measured blood flow velocity associated with a quality value below a threshold quality value.

8. The method of claim 2, wherein generating the functional flow map comprises combining blood pressure or blood flow velocity measurements obtained using the sensor with flow velocity estimates derived from an external ultrasound system.

9. The method of claim 1, wherein using the sensor to obtain the functional flow measurements comprises transmitting and receiving intraluminal ultrasound signals at the sensor.

10. The method of claim 1, further comprising displaying an image including the functional flow map overlaid onto an image of the bodily structure.

11. A system comprising:
an intraluminal device configured for insertion into a bodily structure within a tracked field;
a sensor positioned on the intraluminal device, wherein the sensor is configured to obtain one or more functional flow measurements and configured to receive a signal or cause a disturbance in the tracked field;
a tracking system communicatively coupled to the sensor to generate tracking data responsive to the received signal or disturbance caused by the sensor; and
one or more processors in communication with the sensor and the tracking system, the one or more processors configured to:
use the received signal or disturbance of the sensor to track one or more positions of the intraluminal device within the bodily structure;
use the sensor to obtain the functional flow measurements at the tracked position; and
generate a functional flow map of the bodily structure based on the tracked positions and functional flow measurements.

12. The system of claim 11, wherein the functional flow measurements comprise at least one of blood pressure or blood flow velocity.

13. The system of claim 11, further comprising a display in communication with the one or more processors, wherein the one or more processors are configured to cause the display to display an image including the functional flow map overlaid onto an image of the bodily structure.

14. The system of claim 12, wherein the one or more processors are configured to cause the display to display an indication of a quality of the blood pressure or blood flow velocity measurements obtained using the sensor, and wherein the indication of quality of the blood pressure or blood flow velocity measurements is based, at least in part, on the tracked position.

15. The system of claim 12, wherein the one or more processors are configured to ignore blood pressure or blood flow velocity measurements associated with a quality value below a threshold quality value when generating the functional flow map.

16. The system of claim 11, wherein the tracking system is an ultrasonic tracking system comprising an ultrasound transmitter configured to transmit ultrasound toward the sensor, wherein the sensor includes an ultrasound receiver, and wherein the ultrasonic tracking system is configured to localize the position of the ultrasound receiver by performing one-way beamforming of signals received by the ultrasound receiver when placed within a field of view of the ultrasound transmitter.

17. The system of claim 16, wherein the tracking system is provided by an ultrasonic array of an ultrasound imaging system configured to generate an ultrasound image including a B-mode image of the bodily structure overlaid with the functional flow map.

18. The system of claim 17 wherein the ultrasound imaging system is configured to generate an ultrasound image further including the tracked position of the sensor, updated in real-time, in the ultrasound image.

19. The system of claim 11, wherein the tracking system is an ultrasound system configured to generate color-flow Doppler or vector flow images, and wherein the one or more processors are configured to combine the one or more functional flow measurements obtained using the sensor with flow velocity estimates derived by the ultrasound system to generate the functional flow map.

20. The system of claim 11, wherein the tracking system is an electromagnetic (EM) tracking system comprising a field generator configured to generate an EM field, wherein the sensor includes one or more sensor coils, and wherein the EM tracking system is configured to localize the tracked position of the one or more sensor coils responsive to a disturbance of the EM field caused by the one or more sensor coils when placed within the EM field.
